(19) United States
(12) Patent Application Publication
(10) Pub. No.: US 2023/0158041 A1
(43) Pub. Date: May 25, 2023

(54) METHODS AND COMPOSITIONS FOR TREATING AUTOSOMAL DOMINANT POLYCYSTIC KIDNEY DISEASE

(71) Applicant: OTSUKA PHARMACEUTICAL CO.,LTD., Tokyo (JP)

(72) Inventor: Susan Elizabeth Shoaf, Silver Spring, MD (US)

(21) Appl. No.: 17/533,061

(22) Filed: Nov. 22, 2021

(51) Int. Cl. A61K 31/55; A61K 31/505; A61K 31/635; A61P 13/12

(52) U.S. Cl. CPC A61K 31/55; A61K 31/505; A61K 31/635; A61P 13/12

(57) ABSTRACT

Disclosed are methods of treating autosomal dominant polycystic kidney disease (ADPKD) involving administration of a therapeutically effective amount of tolvaptan to a patient in need thereof, wherein said patient also receives or is administered at least one BCRP substrate, (OAT)3 substrate, (OATP)1B1 substrate, or (OATP)1B3 substrate. Also disclosed are methods of treating a patient, the method comprising administering at least one BCRP substrate, (OAT)3 substrate, (OATP)1B1 substrate, or (OATP)1B3 substrate in the presence of a tolvaptan metabolite, e.g., DM-4103. Also disclosed are compositions which contain tolvaptan and one or more substrates selected from BCRP substrates, (OAT)3 substrates, (OATP)1B1 substrates, and (OATP)1B3 substrates.

### CROSS-REFERENCE TO RELATED APPLICATIONS

This field intentionally left blank.

### TECHNICAL FIELD

The subject matter described herein relates to methods of treating autosomal dominant polycystic kidney disease (ADPKD) involving administration of a therapeutically-effective amount of tolvaptan to a patient in need thereof, wherein said patient also receives or is administered at least one breast cancer resistance protein (BCRP) substrate, organic anion transporter 3 ((OAT)3) substrate, OAT protein 1B1 ((OATP)1B1) substrate, or OAT protein 1B3 ((OATP)1B3) substrate. The subject matter described herein also relates to methods of treating a patient, the method comprising administering at least one BCRP substrate, (OAT)3 substrate, (OATP)1B1 substrate, or (OATP)1B3 substrate in the presence of a tolvaptan metabolite, e.g., the oxobutyric acid metabolite of tolvaptan (DM-4103). The subject matter described herein also relates to compositions which contain tolvaptan and one or more substrates selected from BCRP substrates, (OAT)3 substrates, (OATP)1B1 substrates, and (OATP)1B3 substrates.

### BACKGROUND

Polycystic kidney disease is classified into ADPKD and ARPKD (autosomal recessive polycystic kidney disease). In both types of polycystic kidney disease, many cysts develop in the cortex and medulla of the kidney, leading to kidney dysfunction accompanied by substantial atrophy and fibrosis. As the disease progresses, the kidneys develop hypertrophy, leading to kidney failure requiring dialysis.

ADPKD is a dominantly-inherited, systemic disease characterized by development and growth of multiple renal cysts, which are associated with gradual enlargement of the kidneys. Over a period of, typically, decades, ADPKD results in a progressive loss of renal function and ultimately renal failure, which leaves subjects with dialysis or transplant as the only treatment options.

ADPKD can be diagnosed in utero or in infancy, but diagnosis often happens later in life.

Early signs include renal hyperfiltration, loss of urinary concentrating ability, and hypertension. Subjects with ADPKD experience kidney, abdominal, flank, or back pain from enlargement of the kidneys, cyst hemorrhage, kidney stones, and infections. Significant renal impairment typically occurs in the fourth through seventh decade of life.

To delay the progression of ADPKD, clinicians generally seek to manage ADPKD patients by focusing on controlling secondary conditions, e.g., hypertension. However, some pathophysiology-based therapies (e.g., vasopressin-2-receptor [V2R] antagonists and somatostatin analogs) have shown encouraging results in preclinical and clinical studies.

In view of the general approach to treating ADPKD in patients, which focuses on secondary conditions of the disease, there is thus an ongoing need for more effective, targeted treatment methods.

Tolvaptan, a selective V2R antagonist, blocks vasopressin that promotes the number and growth of kidney cysts.

Tolvaptan was approved by the United States Food and Drug Administration (FDA) as JYNARQUE®, marketed by Otsuka America Pharmaceutical, Inc., for the indication to slow kidney function decline in adults at risk of rapidly progressing ADPKD in 2018. In vitro, tolvaptan was a BCRP inhibitor, whereas DM-4103 was an inhibitor of (OATP)1B1 and (OAT)3. Based on a 2017 FDA draft guidance finalized in 2020, “In Vitro Metabolism- and Transporter-Mediated Drug-Drug Interaction Studies, and Clinical Drug Interaction Studies—Study Design, Data Analysis, and Clinical Implications”, the potential for clinically-relevant inhibition at these transporters was indicated for the highest tolvaptan regimen. Consequently, two postmarketing clinical trials in healthy subjects were required. In Trial 1, 5 mg rosuvastatin calcium (BCRP and (OATP)1B1 substrate) was administered alone, with 90 mg tolvaptan or 48 hours following 7 days of once daily 300 mg tolvaptan (i.e., in the presence of DM-4103). In Trial 2, 40 mg furosemide ((OAT)3 substrate) was administered alone and in presence of DM-4103. For BCRP, rosuvastatin geometric mean ratios (90% confidence intervals [CIs]) for maximum plasma concentration (Cₘₐₓ) were 1.54 (90% CI 1.26-1.88) and for area under the concentration-time curve from time 0 to the time of the last measurable concentration (AUCₜ) were 1.69 (90% CI 1.34-2.14), indicating no clinically meaningful interaction. DM-4103 produced no clinically meaningful changes in rosuvastatin or furosemide concentrations, indicating no inhibition at (OATP)1B1 or (OAT)3. The BCRP prediction assumed the drug dose is completely soluble in 250 ml; tolvaptan has solubility of ˜0.01 g/250 ml. For (OATP)1B1/(OAT)3, if fraction unbound for plasma protein binding (PPB) is less than 1%, then 1% is assumed. DM-4103 has PPB greater than 99.8%. Use of actual drug substance solubility and unbound fraction in plasma would have produced predictions consistent with the clinical results.

The FDA required post-marketing clinical trials following the approval of tolvaptan for the treatment of ADPKD to assess the potential for interaction at breast cancer resistance protein (BCRP), organic anion transport polypeptide (e.g., (OATP)1B1 and organic anion transporter ((OAT)3) transporters. See, for example, the prescribing information for JYNARQUE® (accessible online at www.accessdata.fda.gov/drugsatfda_docs/label/2018/204441lbl.pdf), which instructs against concomitant use of tolvaptan with, inter alia, (OATP)1B1 transporter substrates, transporter substrates, (OAT)3 transporter substrates, and BCRP transport substrates.

Additionally, in 2020, the FDA finalized a guidance document intended to help drug developers determine the in vivo drug-to-drug interaction (DDI) potential of an investigational drug product. The earlier draft version released in 2017 contained almost identical language with regard to the prediction of the potential for in vivo inhibition at BCRP, (OATP)1B1, and (OAT)3 transporters. The FDA guidance is also consistent with guidelines published by the European Medicines Agency in 2013 [Guideline on the Investigation of Drug Interactions, European Medicines Agency, 21 Jun. 2012, CPMP/EWP/560/95/Rev. 1 Con. 2**, Committee for Human Medicinal Products (CHMP)—finalized 2013. <https://www.ema.europa.eu/en/documents/scientific-guide line/guide line-investigation-drug-interactions-revision-1_en.pdf.] and the Ministry of Labor and Welfare in Japan in 2018 [Ministry of Labor and Welfare. Guideline on drug interaction for drug development and appropriate provision of information, notification No. 0723-4, pharmaceutical evaluation division, pharmaceuticals safety and environmental Health bureau, Japan. Jul. 23, 2018. https://www.pmda.go.jp/files/00022 8122.pdf. Jan. 20, 2021.].

It has thus been understood by person of ordinary skill in the art, therefore, that tolvaptan should not be administered with (OATP)1B1 transporter substrates, (OAT)3 transporter substrates, or BCRP transport substrates.

### BRIEF SUMMARY

The inventors disclose herein various embodiments of the method of the present invention.

The present invention relates to a method for treating polycystic kidney disease in a patient taking breast cancer resistance protein (BCRP) substrate, comprising administering tolvaptan or a prodrug thereof to a polycystic kidney disease patient.

The present invention also relates to a method for treating polycystic kidney disease in a patient taking organic anion transport (OAT) polypeptide substrate, comprising administering tolvaptan or a prodrug thereof to a polycystic kidney disease patient.

The present invention also relates to a method for treating polycystic kidney disease in a patient taking an organic anion transporter 3 ((OAT)3) substrate, comprising administering tolvaptan or a prodrug thereof to a polycystic kidney disease patient.

The present invention also relates to a method comprising administering to a patient in need thereof a clinically meaningful amount of tolvaptan and a clinically meaningful amount of a substrate selected from (OAT)3 substrates, (OATP)1B1 substrates, and (OATP)1B3 substrates, wherein the tolvaptan and the substrate are co-administered.

The present invention also relates to a method comprising administering to a patient in need thereof a clinically meaningful amount of tolvaptan and a clinically meaningful amount of a BCRP substrate, wherein the tolvaptan and the BCRP substrate are administered no more than ten hours apart.

Other features of the present invention will be made apparent by the following disclosure and the accompanying figures. A person of ordinary skill in the art will appreciate that the presently disclosed embodiments and features are not limiting.

### BRIEF DESCRIPTION OF THE DRAWINGS

FIG. 1 illustrates the designs of Trial 1 and Trial 2, discussed in the Example set forth herein.

FIG. 2 is a plot of the mean (SD) rosuvastatin plasma concentrations measured in Trial 1.

FIG. 3 is a plot of the mean (SD) furosemide plasma concentrations measured in Trial 2.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art. The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

Unless otherwise defined, all technical and scientific terms used herein have the same meaning as commonly understood by a person having ordinary skill in the art to which the present invention belongs. While methods and materials similar or equivalent to those described herein can be used to practice the invention, suitable methods and materials are described herein. All publications, patent applications, patents, and/or other references mentioned herein are incorporated by reference in their entireties. In the event that any of the publications, patent applications, patents and/or other references mentioned and incorporated herein contradict the present disclosure, the present disclosure including the definitions is authoritative. Additionally, the materials, methods, and examples are illustrative only and are not intended to be limiting. The doses and protocols described herein are exemplary only, and the dosages, treatment protocol, and means of administration may vary in other exemplary uses of the methods.

The articles “a” and “an” are used herein to refer to one or to more than one (i.e. to at least one) of the grammatical object of the article. By way of example, an element means one element or more than one element.

Definitions set forth herein refer to the particular term defined as well as all grammatical variations of the particularly defined terms.

As used herein in reference to a value, the term “about” refers to a value that is similar, in context to the referenced value. In general, those skilled in the art, familiar with the context, will appreciate the relevant degree of variance encompassed by “about” in that context. For example, in some embodiments, the term “about” can encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value. The details of one or more embodiments of the invention are set forth in the accompanying drawings and the description below. Further features, objects and advantages of the invention will become apparent from the description and the drawings as well as from the claims.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present disclosure. At the very least, and not as an attempt to limit the application of the doctrine of equivalents to the scope of the claim, each numerical parameter should at least be construed in light of the number of reported significant digits and by applying ordinary rounding techniques.

Whenever a numerical range of degree or measurement with a lower limit and an upper limit is disclosed, any number and any range falling within the range is also intended to be specifically disclosed. For example, every range of values (in the form “from a to b,” or “from about a to about b,” or “from about a to b,” “from approximately a to b,” and any similar expressions, where “a” and “b” represent numerical values of degree or measurement) is to be understood to set forth every number and range encompassed within the broader range of values. All numerical ranges defined herein are inclusive of endpoints and all values thereinbetween, unless otherwise specifically stated. For example, “at a concentration of a-b” means “at a concentration of at least a, and at most b.”

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration” or “to administer” or grammatical variations thereof mean the step of giving (i.e. administering) a pharmaceutical composition to a subject, or alternatively a subject receiving a pharmaceutical composition. The pharmaceutical compositions disclosed herein can be administered orally, for example.

“ADPKD” denotes autosomal dominant polycystic kidney disease.

“Agent” refers to a substance, entity or complex, combination, mixture or system, or phenomenon (e.g., heat, electric current or field, magnetic force or field, etc.). For example, a flavoring agent is a substance imparting flavor to a composition. As used herein, the terms “agent” and “ingredient” are used synonymously and interchangeably.

“Alleviating” means a reduction in a symptom or cause of a condition or disorder. Thus, alleviating includes some reduction, significant reduction, near total reduction, and total reduction.

“Amelioration” refers to the prevention, reduction or palliation of a state, or improvement of the state of a subject. Amelioration includes, but does not require, complete recovery or complete prevention of a disease, disorder or condition.

As used herein, “associated with” denotes a relationship between two events, entities and/or phenomena. Two events, entities and/or phenomena are “associated” with one another, as that term is used herein, if the presence, level and/or form of one is correlated with that of the other. For example, a particular entity is considered to be associated with a particular disease, disorder, or condition, if its presence, level and/or form correlates with incidence of and/or susceptibility to the disease, disorder, or condition (e.g., across a relevant population).

“BCRP” means breast cancer resistance protein, encoded in humans by gene ABCG2, which is an ATP-binding cassette efflux transporter.

“BCRP substrate” means a pharmaceutically active ingredient or composition containing a pharmaceutically active ingredient which is a substrate for BCRP. Exemplary, non-limiting BCRP substrates include anthracenes such as mitoxantrone, bisantrene, and aza-anthrapyrazole (BBR3390); camptothecin derivates such as topotecan, SN-38, irinotecan, and diflomotecan; polyglutamates such as methotrexate, methotrexate-glu2, and methotrexate-glu3; nucleoside analogs such as AZT, AZT 5′-monophosphate, and lamivudine (3TC); other drugs, such as prazosin, indolocarbazole, flavopiridol, and canertinib (CI1033), imatinib mesylate (STI571), gefitinib (ZD1839), nilotinib, glyburide, cimetidine, sulfasalazine, nitrofurantoin, rosuvastatin, and pantoprazole; chemical toxicants such as the carcinogen 2-amino-1-methyl-6-phenylimidazo[4,5-b]pyridine (PhIP); phototoxic compounds such as protoporphyrin ix, the lipid phosphatidylserine; and flavonoids such as genistein, uric acid, and vitamins.

As used herein, the term “clinically meaningful dose” or “clinically meaningful amount” or the like means a dose or amount of a pharmaceutically active ingredient or composition which delivers a pharmaceutically active ingredient, which alleviates, reduces, eliminates or otherwise treats at least one symptom of a disease or condition and wherein the dose or amount comports or substantially comports with clinically approved doses or amounts or other doses or amounts as would be prescribed by a physician.

As used herein, the term “clinically-meaningful effect” means an effect on a dosing regimen, treatment method, or other protocol or paradigm for treating one or more symptoms, conditions or diseases, which a person of ordinary skill in the art would consider to have a significant effect on the efficacy of the regimen/method/protocol/paradigm, or which a person of ordinary skill in the art would regard as introducing one or more significant adverse effects or toxicity. For example, and without limitation, where co-administration of A and B results in meaningfully impaired effect of A as compared to administration of A alone, a person of ordinary skill in the art would regard such impairment as a clinically-meaningful effect.

“Effective amount” as applied to the biologically active ingredient means that amount of the ingredient that is generally sufficient to effect a desired change in the subject. For example, where the desired effect is a reduced decline rate of estimated glomerular filtration rate (eGFR), an effective amount of the ingredient is that amount that causes a substantial reduction in the rate of loss of eGFR, without resulting in significant toxicity.

“eGFR” as used herein, refers to the estimated glomerular filtration rate in patients with autosomal dominant polycystic kidney disease. As used herein, “eGFR” means a value descriptive of estimated glomerular filtration rate as calculated according to the Chronic Kidney Disease Epidemiology Collaboration (CKD-EPI) equation, which calculates eGFR based on serum creatine, age, sex and race.

“Glomerular” refers to a network of capillaries known as a tuft, located at the beginning of a nephron in the kidney. The glomerulus is a loop of capillaries twisted into a ball shape, surrounded by the Bowman's capsule, which is where ultrafiltration of blood occurs (i.e., the first step in urine production).

“(OAT)3” refers to solute carrier family 22 member 8, or organic anion transporter 3 (i.e., (OAT)3), which is a protein that, in humans, is encoded by the SLC22A8 gene.

“(OAT)3 substrate” means a pharmaceutically active ingredient or composition containing a pharmaceutically active ingredient which is a substrate for (OAT)3. Exemplary, non-limiting (OAT)3 substrates include acamprosate, acyclovir, allopurinol, alprostadil, aminohippuric acid, avibactam, baricitinib, benzylpenicillin, betamethasone phosphate, budesonide, bumetanide, captopril, cefacetrile, cefaclor, cefaloridine, cefazolin, cefdinir, cefotiam, ceftibuten, ceftizoxime, cephalexin, cholic acid, cimetidine, citrulline, clofarabine, cloxacillin, conjugated estrogens, cortisone acetate, cyclic adenosine monophosphate, dexamethasone, dexamethasone acetate, dinoprostone, doripenem, edaravone, ellagic acid, eluxadoline, empagliflozin, estradiol, estrone, famotidine, fexofenadine, fluorescein, glutaric acid, hydrochlorothiazide, hydrocortisone, hydroflumethiazide, indomethacin, leucovorin, levocarnitine, medrysone, mercaptopurine, methotrexate, oseltamivir, oxalic acid, oxytetracycline, pemetrexed, piperacillin, polythiazide, pravastatin, prednisolone phosphate, quinapril, ranitidine, relebactam, rosuvastatin, saxagliptin, silibinin, sitagliptin, succinic acid, taurocholic acid, tazobactam, tenofovir, tenofovir alafenamide, tenofovir disoproxil, tetracycline, trifluridine, valaciclovir, valproic acid, and zidovudine.

“(OATP)1B1” means organic anion transporting polypeptide 1B1, encoded by solute carrier organic anion transporter family member 1B1 (SLCO1B1).

“(OATP)1B1 substrate” means a pharmaceutically active ingredient or composition containing a pharmaceutically active ingredient which is a substrate for (OATP)1B1. Exemplary, non-limiting (OATP)1B1 substrates include ambrisentan, asunaprevir, atorvastatin, atrasentan, axitinib, belantamab mafodotin, belzutifan, benzylpenicillin, bosentan, brincidofovir, caspofungin, cerivastatin, cholecystokinin, cholic acid, clotrimazole, cobimetinib, conjugated estrogens, cyclosporine, digoxin, dinoprostone, elagolix, eluxadoline, empagliflozin, enalapril, erythromycin, ezetimibe, fexofenadine, fimasartan, fluvastatin, gadoxetic acid, gimatecan, glecaprevir, grazoprevir, letermovir, levomenol, levosalbutamol, liothyronine, liotrix, lovastatin, methotrexate, mycophenolate mofetil, olmesartan, ouabain, paritaprevir, penicillamine, pitavastatin, prasterone, pravastatin, raloxifene, remdesivir, repaglinide, revefenacin, rosuvastatin, selexipag, simeprevir, simvastatin, sumatriptan, taurocholic acid, technetium tc-99 m mebrofenin, temocapril, tenofovir alafenamide, torasemide, ubrogepant, valsartan, and voxilaprevir.

“(OATP)1B3” means organic anion transporting polypeptide 1B3, encoded by solute carrier organic anion transporter family member 1B3 (SLCO1B3).

“(OATP)1B3 substrate” means a pharmaceutically active ingredient or composition containing a pharmaceutically active ingredient which is a substrate for (OATP)1B3. Exemplary, non-limiting (OATP)1B3 substrates include ambrisentan, atogepant, atorvastatin, belantamab mafodotin, belzutifan, bempedoic acid, brincidofovir, caspofungin, cholecystokinin, cholic acid, cobimetinib, conjugated estrogens, docetaxel, empagliflozin, erythromycin, fexofenadine, fluvastatin, gadoxetic acid, glecaprevir, grazoprevir, letermovir, levomenol, levosalbutamol, liothyronine, liotrix, methotrexate, mycophenolate mofetil, olmesartan, opicapone, ouabain, paclitaxel, parachlorophenol, paritaprevir, pitavastatin, prasterone, pravastatin, raloxifene, revefenacin, rifampicin, romidepsin, selexipag, simeprevir, sincalide, taurocholic acid, technetium tc-99 m mebrofenin, tenofovir alafenamide, testosterone, trastuzumab deruxtecan, ubrogepant, valsartan, and voxilaprevir.

“Patient” means a human or non-human subject receiving medical or veterinary care. Accordingly, the compositions as disclosed herein can be used in treating any animal, such as, for example, mammals, or the like.

“Pharmaceutically-acceptable” or “therapeutically-acceptable” as applied to any carrier, diluent, or other additive or excipient used to formulate a composition as disclosed herein means that the carrier, diluent, additive or other excipient is compatible with the other ingredients contained in the composition and is not deleterious to the recipient thereof. Similarly, as used herein, the term “pharmaceutically-acceptable carrier” or “therapeutically-acceptable carrier” means a pharmaceutically- or therapeutically-material, composition or vehicle, such as a liquid or solid filler, diluent, excipient, or solvent-encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

“Pharmaceutical composition” or “composition” means a composition comprising an active pharmaceutical ingredient, such as, for example, tolvaptan, and at least one additional ingredient, such as, for example, a stabilizer or excipient or the like. A pharmaceutical composition is therefore a formulation that is suitable for diagnostic or therapeutic administration to a subject, such as a human patient. The pharmaceutical composition can be, for example, a tablet for oral use. Those skilled in the art will appreciate that the term “composition”, as used herein, can be used to refer to a discrete physical entity that comprises one or more specified components. In general, unless otherwise specified, a composition can be of any form, e.g., gas, gel, liquid, solid, etc.

As used herein when used in connection with the occurrence of a disease, disorder, and/or condition, “prevent” (and grammatical variations thereof) refers to reducing the risk of developing the disease, disorder and/or condition and/or to delaying onset of one or more characteristics or symptoms of the disease, disorder or condition. Prevention can be considered complete when onset of a disease, disorder or condition has been delayed for a predefined period of time.

“ADPKD-management method” or “ADPKD management” or “ADPKD treatment” or “ADPKD-treatment method” are used interchangeably herein and each refers to the treatment accepted by persons of ordinary skill in the art, such as medical experts and practitioners, at the time the inventors have made the present disclosure. More specifically, in some embodiments of the present invention, “ADPKD management” and each of the above equivalent terms refers to either or both of (a) one or more symptomatic treatments of ADPKD-related disease features, such as controlling blood pressure, pain-relief medications and pain-relief interventions, and/or (b) one or more generally accepted measures believed to be supportive to maintaining kidney health, such as increasing water intake, modifying diet, and/or controlling blood pressure.

“Substantially free,” as used in connection or relation to a substance or property, refers to quantities of less than about 1% of such substance or property, preferably less than about 0.1% for the indicated substance or property.

“Tolvaptan” refers to 7-chloro-5-hydroxy-1-[2-methyl-4-(2-methylbenzoylamino)benzoyl]-2,3,4,5-tetrahydro-1H-benzoazepine, represented by Formula (1):

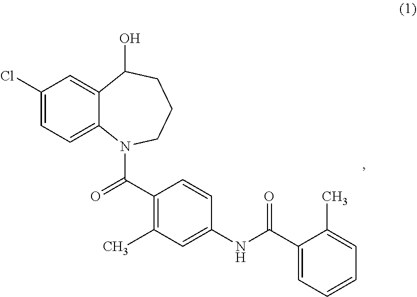 [SMILES: Cc1ccccc1C(=O)Nc1ccc(C(=O)N2CCCC(O)c3cc(Cl)ccc32)c(C)c1]

including crystalline forms of a compound of Formula (1), amorphous forms of a compound of Formula (1), and/or anhydrides, solvates (e.g., hydrates, alcoholates. etc.), salts, and/or co-crystals of a compound of Formula (1), and combinations or mixtures thereof. As used herein, “tolvaptan” includes prodrugs obtained by modifying a compound of Formula (1). Additionally, as used herein, “tolvaptan” includes compounds according to Formula (1) in which one or more atoms are replaced by one or more isotopes thereof (e.g., deuterium, tritium, carbon-13, nitrogen-14, oxygen-18, and the like). Furthermore, unless otherwise specified, “tolvaptan” as used herein means either the R- or S-enantiomer of the compound of Formula (1) based on the hydroxyl-bonded asymmetric carbon. Additionally, “tolvaptan” includes, unless otherwise specified, a racemic mix of the R- and S-enantiomers of the compound of Formula (1).

“Total kidney volume” or “TKV” means the cumulative or overall renal volume in a patient, as measured by magnetic resonance imaging.

“Treatment” (and grammatical variations thereof) refers to administration of a therapy that partially or completely alleviates, ameliorates, relieves, inhibits, delays onset of, reduces severity of, and/or reduces incidence of one or more symptoms, features, and/or causes of a particular disease, disorder, and/or condition, or is administered for the purpose of achieving any such result. In some embodiments, such treatment can be of a subject who does not exhibit signs of the relevant disease, disorder and/or condition and/or of a subject who exhibits only early signs of the disease, disorder, and/or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, and/or condition.

In various examples, treatment is of ADPKD.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

A pharmaceutical composition described herein can also include preservative agents such as benzyl alcohol, benzoic acid, phenol, parabens and sorbic acid. Pharmaceutical compositions can include, for example, excipients, such as surface active agents; dispersing agents; inert diluents; granulating and disintegrating agents; binding agents; lubricating agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; antioxidants; stabilizing agents; and pharmaceutically acceptable polymeric or hydrophobic materials and other ingredients known in the art and described, for example in Genaro, ed., 1985, Remington's Pharmaceutical Sciences, Mack Publishing Co., Easton, Pa., which is incorporated herein by reference.

In other embodiments, the ADPKD management method is one or more generally-accepted measures believed to be supportive to maintaining kidney health, such as increasing water intake, modifying diet, and/or controlling blood pressure. In a surprising aspect of the present invention, an ADPKD management method may be employed or administered in combination with a substrate of BCRP, (OATP)1B1/3 or (OAT)3 without a clinically-meaningfully effect on the substrate's efficacy.

In other embodiments, the ADPKD management method is a combination of (a) one or more symptomatic treatments of ADPKD-related disease features, such as controlling blood pressure, pain-relief medications and pain-relief interventions, and (b) one or more generally accepted measures believed to be supportive to maintaining kidney health, such as increasing water intake, modifying diet, and/or controlling blood pressure. Said ADPKD management method may be employed or administered in combination with a substrate of BCRP, (OATP)1B1/3 or (OAT)3 without a clinically-meaningfully effect on the substrate's efficacy.

In other embodiments, the ADPKD management method is any management or treatment method accepted by persons of ordinary skill in the art (e.g., medical experts and practitioners) at the time the inventors have made the present disclosure. In an aspect, the ADPKD management method is a method of managing one or more symptoms of ADPKD over time. Said ADPKD management method may be employed or administered in combination with a substrate of BCRP,

Described below are non-limiting exemplary embodiments of compositions and methods of treatment involving administering said compositions to ADPKD patients. Additional aspects of the compositions and methods of the present invention will become apparent to persons of ordinary skill in the art based on the disclosure provided herein.

### II. Compositions

Compositions comprising tolvaptan for treatment of ADPKD are described herein. In an aspect, a composition described herein may comprise any pharmaceutically-acceptable additive or excipient.

For example and without limitation, a composition described herein may contain one or more binders, one or more glidants, lubricants, disintegrants or superdisintegrants, sweetening agents or flavoring agents or palatants, pigments or colorants, wetting agents, humectants, and/or one or more preservative agents, and/or other pharmaceutically acceptable excipients or additives.

In another aspect, a composition described herein is formulated for oral administration.

In certain embodiments, the tolvaptan-containing composition is a coated or uncoated tablet that may be chewable or non-chewable. In other embodiments, the tolvaptan-containing composition is a capsule, lozenge, pill, pastille, granule, powder, liquid, concentrated syrup, or other pharmaceutical dosage form.

In certain embodiments, the composition comprises tolvaptan in an amount of about 1 mg to about 120 mg, or about 5 mg to about 100 mg, or about 10 mg to about 90 mg.

In certain embodiments, the composition is a tablet, which comprises tolvaptan in an amount of about 15 mg, or about 30 mg, or about 45 mg, or about 60 mg, or about 90 mg, or about 120 mg.

In certain embodiments, tolvaptan is the sole active biologically active agent or ingredient present in the composition.

In certain embodiments of the method described below, the tolvaptan-containing composition is the only ADPKD-treating composition administered. In other embodiments, the tolvaptan-containing composition is administered in combination with one or more other medicaments.

In a preferred embodiment, the tolvaptan-containing composition is a non-chewable tablet.

In other embodiments, tolvaptan is provided with at least one substrate of BCRP, (OAT)3, or (OATP)1B1, or (OATP)1B3 in a combination formulation.

In certain embodiments of a once-daily treatment, a composition is provided which is formulated as an extended-release formulation. In other embodiments, the once-daily formulation is an immediate-release formulation.

Compositions described herein may further contain a disintegrant. Non-limiting examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. In a preferred embodiment, compositions described herein contain hydroxypropyl cellulose.

Compositions described herein may further contain a glidant. Exemplary glidants include, without limitation, colloidal silica, corn starch and talc.

Compositions described herein may further contain a wetting agent or dispersant. Exemplary wetting agents and dispersants include, without limitation, heptadecaethylene oxycetanol, lecithins, sorbitol monooleate, polyoxyethylene sorbitol monooleate, and polyoxyethylene stearate.

Compositions described herein may further contain an emulsifying agent. Exemplary emulsifying agents include, without limitation, naturally occurring gums, naturally-occurring phosphatides (e.g., soy bean or lecithin), esters or partial esters derived from fatty acids and/or hexitol anhydrides, (e.g., sorbitan monooleate), (4) condensation products of said partial esters with ethylene oxide, (e.g., polyoxyethylene sorbitan monooleate).

Compositions described herein may optionally contain a flavorant or palatant, and/or a sweetening agent.

Compositions described herein may further contain a lubricant. Exemplary lubricants include, without limitation, calcium stearate, magnesium stearate, mineral oil, stearic acid and zinc stearate.

Compositions described herein may further contain a binder. Exemplary binders include, without limitation, acacia, alginic acid, carboxymethylcellulose sodium, compressible sugar, ethylcellulose, gelatin, liquid glucose, methylcellulose, non-crosslinked polyvinyl pyrrolidone, and pregelatinized starch.

Compositions described herein may further contain a coating agent. Exemplary coating agents include, without limitation, liquid glucose, hydroxyethyl cellulose, hydroxypropyl cellulose, hydroxypropyl methylcellulose, methylcellulose, ethylcellulose, cellulose acetate phthalate and shellac.

Additionally, tablets described herein may further contain a colorant.

Tablet dosage forms according to the present disclosure preferably contain corn starch, lactose monohydrate, magnesium stearate and microcrystalline cellulose.

### III. Methods of Treatment

Methods of treating ADPKD are described. The methods comprise administering a composition comprising a pharmaceutically-acceptable amount of tolvaptan to a subject in accord with an administration paradigm determined based on the clinical presentation of the subject. In particular, methods of treating ADPKD and one or more other conditions, symptoms or diseases are described.

Contrary to the FDA's guidance and requirements with respect to the JYNARQUE® initial prescribing information, it has now been found that tolvaptan and a substrate such as BCRP substrate, (OATP)1B1 substrate, (OATP)1B3 substrate, and/or (OAT)3 substrate can be co-administered in a treatment paradigm without having any clinically-meaningful effect on the administered substrate, as would be expected based on the FDA's guidance and findings.

Also contrary to the FDA's guidance and requirements with respect to the initial JYNARQUE® prescribing information, it has likewise been found that a substrate such as BCRP substrate, (OATP)1B1 substrate, (OATP)1B3 substrate, and/or (OAT)3 substrate can be administered in the presence of tolvaptan metabolite without any clinically-meaningful effect on the administered substrate, as would be expected based on the FDA's guidance and findings. In certain embodiments, the tolvaptan metabolite is DM-4103.

For example and without limitation, the present disclosure provides for methods of treating ADPKD and one or more conditions, symptoms or diseases associated with BCRP.

Additionally, and without limitation, the present disclosure also provides for methods of treating ADPKD and one or more conditions, symptoms or diseases associated with (OAT)3.

Additionally, and without limitation, the present disclosure also provides for methods of treating ADPKD and one or more conditions, symptoms or diseases associated with (OATP)1B1.

Additionally, and without limitation, the present disclosure also provides for methods of treating ADPKD and one or more conditions, symptoms or diseases associated with (OATP)1B3.

In an embodiment, the treatment method described herein involves administration of a composition comprising a pharmaceutically-acceptable amount of tolvaptan to a subject, wherein said subject is undergoing treatment for one or more conditions in addition to ADPKD, wherein said treatment for said one or more conditions involves administration of a BCRP substrate. In certain embodiments, administration of the BCRP substrate occurs contemporaneously with tolvaptan (i.e., co-administration). In other embodiments, administration of tolvaptan and administration the BCRP substrate occur within 30 minutes, within one hour, within two hours, within three hours, within four hours, within five hours, within six hours, within eight hours, within nine hours, or within ten hours.

In an aspect, the patient has been diagnosed with a condition or disease for which a BCRP substrate has been prescribed. In some embodiments, the patient has been diagnosed with a cancer and is undergoing treatment involving, inter alia, administration of at least one BCRP substrate.

In an aspect, any BCRP substrate may be administered according to the present method. Preferred, non-limiting examples of BCRP substrates include anthracenes such as mitoxantrone, bisantrene, and aza-anthrapyrazole (BBR3390); camptothecin derivates such as topotecan, SN-38, irinotecan, and diflomotecan; polyglutamates such as methotrexate, methotrexate-glu2, and methotrexate-glu3; nucleoside analogs such as AZT, AZT 5′-monophosphate, and lamivudine (3TC); other drugs, such as prazosin, indolocarbazole, flavopiridol, and canertinib (CI1033), imatinib mesylate (STI571), gefitinib (ZD1839), nilotinib, glyburide, cimetidine, sulfasalazine, nitrofurantoin, rosuvastatin, and pantoprazole; chemical toxicants such as the carcinogen 2-amino-1-methyl-6-phenylimidazo[4,5-b]pyridine (PhIP); phototoxic compounds such as protoporphyrin ix, the lipid phosphatidylserine; and flavonoids such as genistein, uric acid, and vitamins.

In an embodiment, the treatment method described herein involves administration of a composition comprising a pharmaceutically-acceptable amount of tolvaptan to a subject, wherein said subject is undergoing treatment for one or more conditions in addition to ADPKD, wherein said treatment for said one or more conditions involves administration of an (OAT)3 substrate. In certain embodiments, administration of the (OAT)3 substrate occurs contemporaneously with tolvaptan (i.e., co-administration). In other embodiments, administration of tolvaptan and administration the (OAT)3 substrate occur within 30 minutes, within one hour, within two hours, within three hours, within four hours, within five hours, within six hours, within nine hours, within twelve hours, within 24 hours, within 36 hours, within 48 hours, within 72 hours, or within 96 hours of one another.

In an aspect, the patient has been diagnosed with a condition or disease for which an (OAT)3 substrate has been prescribed. In some embodiments, the patient has been diagnosed with a cancer and is undergoing treatment involving, inter alia, administration of at least one (OAT)3 substrate.

In an aspect, any (OAT)3 substrate may be administered according to the present method. Preferred, non-limiting examples of (OAT)3 substrates include acamprosate, acyclovir, allopurinol, alprostadil, aminohippuric acid, avibactam, baricitinib, benzylpenicillin, betamethasone phosphate, budesonide, bumetanide, captopril, cefacetrile, cefaclor, cefaloridine, cefazolin, cefdinir, cefotiam, ceftibuten, ceftizoxime, cephalexin, cholic acid, cimetidine, citrulline, clofarabine, cloxacillin, conjugated estrogens, cortisone acetate, cyclic adenosine monophosphate, dexamethasone, dexamethasone acetate, dinoprostone, doripenem, edaravone, ellagic acid, eluxadoline, empagliflozin, estradiol, estrone, famotidine, fexofenadine, fluorescein, glutaric acid, hydrochlorothiazide, hydrocortisone, hydroflumethiazide, indomethacin, leucovorin, levocarnitine, medrysone, mercaptopurine, methotrexate, oseltamivir, oxalic acid, oxytetracycline, pemetrexed, piperacillin, polythiazide, pravastatin, prednisolone phosphate, quinapril, ranitidine, relebactam, rosuvastatin, saxagliptin, silibinin, sitagliptin, succinic acid, taurocholic acid, tazobactam, tenofovir, tenofovir alafenamide, tenofovir disoproxil, tetracycline, trifluridine, valaciclovir, valproic acid, and zidovudine.

In an embodiment, the treatment method described herein involves administration of a composition comprising a pharmaceutically acceptable amount of tolvaptan to a subject, wherein said subject is undergoing treatment for one or more conditions in addition to ADPKD, wherein said treatment for said one or more conditions involves administration of an (OATP)1B1 or (OATP)1B3 substrate. In certain embodiments, administration of the (OATP)1B1 or (OATP)1B3 substrate occurs contemporaneously with tolvaptan (i.e., co-administration). In other embodiments, administration of tolvaptan and administration the (OATP)1B1 or (OATP)1B3 substrate occur within 30 minutes, within one hour, within two hours, within three hours, within four hours, within five hours, within six hours, within nine hours, within twelve hours, within 24 hours, within 36 hours, within 48 hours, within 72 hours, or within 96 hours of one another.

In an aspect, the patient has been diagnosed with a condition or disease for which an (OATP)1B1 substrate or an (OATP)1B3 substrate has been prescribed. In some embodiments, the patient has been diagnosed with a cancer and is undergoing treatment involving, inter alia, administration of at least one (OATP)1B1 substrate or at least one (OATP)1B3 substrate.

In an aspect, any (OATP)1B1 substrate or any (OATP)1B3 substrate may be administered according to the present method.

Preferred, non-limiting examples of (OATP)1B1 substrates include ambrisentan, asunaprevir, atorvastatin, atrasentan, axitinib, belantamab mafodotin, belzutifan, benzylpenicillin, bosentan, brincidofovir, caspofungin, cerivastatin, cholecystokinin, cholic acid, clotrimazole, cobimetinib, conjugated estrogens, cyclosporine, digoxin, dinoprostone, elagolix, eluxadoline, empagliflozin, enalapril, erythromycin, ezetimibe, fexofenadine, fimasartan, fluvastatin, gadoxetic acid, gimatecan, glecaprevir, grazoprevir, letermovir, levomenol, levosalbutamol, liothyronine, liotrix, lovastatin, methotrexate, mycophenolate mofetil, olmesartan, ouabain, paritaprevir, penicillamine, pitavastatin, prasterone, pravastatin, raloxifene, remdesivir, repaglinide, revefenacin, rosuvastatin, selexipag, simeprevir, simvastatin, sumatriptan, taurocholic acid, technetium tc-99 m mebrofenin, temocapril, tenofovir alafenamide, torasemide, ubrogepant, valsartan, and voxilaprevir.

Preferred, non-limiting examples of (OATP)1B3 substrates include ambrisentan, atogepant, atorvastatin, belantamab mafodotin, belzutifan, bempedoic acid, brincidofovir, caspofungin, cholecystokinin, cholic acid, cobimetinib, conjugated estrogens, docetaxel, empagliflozin, erythromycin, fexofenadine, fluvastatin, gadoxetic acid, glecaprevir, grazoprevir, letermovir, levomenol, levosalbutamol, liothyronine, liotrix, methotrexate, mycophenolate mofetil, olmesartan, opicapone, ouabain, paclitaxel, parachlorophenol, paritaprevir, pitavastatin, prasterone, pravastatin, raloxifene, revefenacin, rifampicin, romidepsin, selexipag, simeprevir, sincalide, taurocholic acid, technetium tc-99 m mebrofenin, tenofovir alafenamide, testosterone, trastuzumab deruxtecan, ubrogepant, valsartan, and voxilaprevir.

In certain embodiments, a method according to the present disclosure involves administering to a subject in need thereof a composition comprising tolvaptan such that the decline in eGFR over time is reduced relative to the eGFR decline resulting from the ADPKD management, and wherein the subject is also undergoing treatment for at least one other disease or condition, wherein said other treatment involves administration of a BCRP substrate, an (OAT)3 substrate, or an (OATP)1B1P1/P3 substrate, or a combination thereof.

In an aspect, when a patient is administered a clinically meaningful dose of at least one BCRP substrate (i.e., an amount of BCRP substrate in accordance with the prescribing information for the BCRP substrate; e.g., 5 mg, or 10 mg, or 15 mg, or 20 mg, or 25 mg, or 30 mg, or 40 mg, or the like) in the presence of tolvaptan metabolite (DM-4103), the lower bounds of the 90% confidence interval for maximum concentration (Cₘₐₓ) and area under the curve (AUC) is less than about 1.0% for BCRP substrate in the presence of tolvaptan metabolite (DM-4103) as compared to the lower bounds of the 90% confidence interval for maximum concentration (Cₘₐₓ) and area under the curve (AUC) when the BCRP substrate is administered alone.

In certain, non-limiting embodiments, the BCRP substrate is rosuvastatin.

In another aspect, when a patient is administered up to about 300 mg tolvaptan daily, there is no clinically meaningful effect on the BCRP substrate administered.

In yet another aspect, when a patient is administered, in a single dose, an amount of tolvaptan ranging from about 50 mg to about 300 mg, there is no clinically meaningful effect on the BCRP substrate administered.

In an aspect, the present inventors surprisingly found that administration of any clinically meaningful dose of tolvaptan would not have any interactions with an (OAT)3 substrate, a BCRP substrate, or an (OATP)1B1/3 substrate.

In another aspect, the present inventors surprisingly found that administration of any clinically meaningful dose of tolvaptan would not have any interactions with any clinically meaningful dose of an (OAT)3 substrate, a BCRP substrate, or an (OATP)1B1/3 substrate.

In an aspect, when a patient is administered a clinically meaningful dose of at least one organic anion transport polypeptide substrate (i.e., an amount of organic anion transport polypeptide substrate in accordance with the prescribing information for the organic anion transport polypeptide substrate; e.g., 5 mg, or 10 mg, or 15 mg, or 20 mg, or 25 mg, or 30 mg, or 40 mg, or the like) in the presence of tolvaptan metabolite (DM-4103), the lower bounds of the 90% confidence interval for maximum concentration (Cmax) and area under the curve (AUC) is less than about 1.0% for the organic anion transport polypeptide substrate substrate in the presence of tolvaptan metabolite (DM-4103) as compared to the lower bounds of the 90% confidence interval for maximum concentration (Cmax) and area under the curve (AUC) when the organic anion transport polypeptide substrate is administered alone.

In certain, non-limiting embodiments, the organic anion transport polypeptide substrate is rosuvastatin.

In another aspect, when a patient is administered up to about 300 mg tolvaptan, up to about 500, or up to about 600 mg tolvaptan daily, there is no clinically-meaningful effect on the organic anion transport polypeptide substrate (e.g., an (OATP)1B1 or (OATP)1B3 substrate) administered.

In yet another aspect, when a patient is administered, in a single dose, an amount of tolvaptan ranging from about 50 mg to about 600 mg, there is no clinically-meaningful effect on the organic anion transport polypeptide substrate (e.g., an (OATP)1B1 or (OATP)1B3 substrate) administered.

In an aspect, when a patient is administered a clinically meaningful dose of at least one (OAT)3 substrate (i.e., an amount of (OAT)3 substrate in accordance with the prescribing information for the (OAT)3 substrate; e.g., 5 mg, or 10 mg, or 15 mg, or 20 mg, or 25 mg, or 30 mg, or 40 mg, or the like) in the presence of tolvaptan metabolite (DM-4103), the lower bounds of the 90% confidence interval for maximum concentration (Cmax) and area under the curve (AUC) is less than about 1.0% for (OAT)3 substrate substrate in the presence of tolvaptan metabolite (DM-4103) as compared to the lower bounds of the 90% confidence interval for maximum concentration (Cmax) and area under the curve (AUC) when the (OAT)3 substrate is administered alone.

In certain, non-limiting embodiments, the (OAT)3 substrate is furosemide.

In another aspect, when a patient is administered up to about 300 mg tolvaptan daily, there is no clinically-meaningful effect on the (OAT)3 substrate administered.

In yet another aspect, when a patient is administered, in a single dose, an amount of tolvaptan ranging from about 50 mg to about 300 mg, there is no clinically-meaningful effect on the (OAT)3 substrate administered.

In certain embodiments of the method disclosed herein, a patient may receive tolvaptan alone on certain days and on other days the patient may receive both tolvaptan and a substrate (i.e., an (OAT)3 substrate, an (OATP)1B1 substrate, an (OATP)1B3 substrate, or any combination thereof). On days which the patient receives tolvaptan alone, the patient may be administered, for example, up to about 600 mg tolvaptan, up to about 500 mg tolvaptan, up to about 400 mg tolvaptan, or up to about 300 mg tolvaptan across one, two or three daily doses. On days which the patient receives tolvaptan and a substrate, the patient may be administered up to about 300 mg tolvaptan, up to about 200 mg tolvaptan, or up to about 100 mg tolvaptan across one, two or three daily doses, and the patient may be administered, for example, about 1-100 mg substrate, or about 2-75 mg substrate, or about 3-60 mg substrate, or any amount therebetween across any number of appropriate doses per day, as prescribed. On days which the patient is administered only substrate, the patient may be administered about 1-100 mg substrate, or about 2-75 mg substrate, or about 3-60 mg substrate, or any amount therebetween. In an aspect, a patient may receive the same amount of substrate regardless of whether the patient receives only substrate or substrate and tolvaptan on a given day.

In certain embodiments of the present invention, a tolvaptan-containing composition is administered to a subject such that patients receive twice per day split dose administration of tolvaptan immediate-release in the range of 45/15 mg, 60/30 mg, 90/30 mg or single, daily doses of AUC equivalent dosage forms with continuous treatment until a patient reaches the need for renal replacement therapy.

In other embodiments, a tolvaptan-containing composition is administered to a subject such that patients receive a total daily dose of tolvaptan in an amount ranging from about 30 mg to about 120 mg. In an aspect, the dosage of tolvaptan can be adjusted downward or down-titrated depending in any interactions between drugs.

In yet other embodiments, a tolvaptan-containing composition is administered to a subject such that patients receive about 60 mg tolvaptan, or about 90 mg tolvaptan, or about 120 mg tolvaptan per day in the form of an immediate-release pharmaceutical composition. In an aspect, the dosage of tolvaptan can be adjusted downward or down-titrated depending in any interactions between drugs.

The ADPKD management, on the other hand, is defined as (a) the treatment of one or more symptomatic treatments of ADPKD-related disease features, such as controlling blood pressure, pain-relief medications and pain-relief interventions, and/or (b) the implementation or administration of one or more generally accepted measures believed to be supportive to maintaining kidney health, such as increasing water intake, modifying diet, and/or controlling blood pressure.

In certain embodiments of the inventive method, a composition containing tolvaptan is administered to a patient once daily, twice daily or three times a day. In certain embodiments, the total amount of tolvaptan administered daily may be up to about 300 mg. Non-limiting administration paradigms are set forth below for purposes of illustration.

In an exemplary, non-limiting twice-daily administration method, a patient may receive, for example, a total amount of tolvaptan of about 30 mg to about 120 mg, or about 45 mg to about 100 mg, or about 60 mg to about 90 mg per day. In certain embodiments of a twice-daily administration method, a patient receives a total amount of tolvaptan of about 60 mg per day. In such embodiments, a first dose delivers about 45 mg tolvaptan and a second dose delivers about 15 mg tolvaptan. In certain other embodiments of a twice-daily administration method, a patient receives a total amount of tolvaptan of about 90 mg per day. In such embodiments, a first dose delivers about 60 mg tolvaptan and a second dose delivers about 30 mg tolvaptan. In yet other embodiments of a twice-daily administration method, a patient receives a total amount of tolvaptan of about 120 mg per day. In such embodiments, a first dose delivers about 90 mg tolvaptan and a second dose delivers about 30 mg tolvaptan. In certain embodiments of a twice-daily administration method, a patient receives two doses about twelve hours apart, about ten hours apart, about eight hours apart, or about six hours apart.

In certain embodiments, the dosage of tolvaptan may be titrated up from a 45 mg first dose and a 15 mg second dose (i.e., 45 mg/15 mg) to 60 mg/30 mg per, and then to 90 mg/30 mg per day if tolerated with at least weekly intervals between titrations.

In certain embodiments of the present method, a patient may down-titrate based on tolerability. For example and without limitation, a patient may down-titrate to a total dosage which is at least 5% less, at least 10% less, at least 15% less, at least 20% less, at least 25% less, at least 30% less, at least 33% less, at least 45% less, or at least 50% less than the initial dosage. For example and without limitation, a dosage may be down-titrated to a total daily dosage of about 120 mg tolvaptan, about 90 mg tolvaptan, 60 mg tolvaptan, 45 mg tolvaptan, or the like.

In yet other preferred embodiments, a once-daily administration method and dosage form is provided. In certain embodiments of a once-daily administration, a patient receives a single dose of up to about 600 mg tolvaptan or up to about 500 mg tolvaptan, or up to about 400 mg tolvaptan or up to about 300 mg tolvaptan. In certain embodiments of a once-daily administration method, a patient receives a single dose which delivers about 15 g to about 600 mg tolvaptan, preferably about 15 mg to about 500 mg tolvaptan, and even more preferably about 30 mg to about 400 mg tolvaptan.

The present inventors surprisingly found that treatment methods according to the present invention achieve reductions in eGFR decline in ADPKD patients significantly greater than reductions in eGFR declines achieved according to the ADPKD management.

For example, in certain embodiments, a reduction in eGFR decline of 0.25 mL/min/1.73 m2 is achieved. In yet other exemplary embodiments, the reduction is at least of 0.33 mL/min/1.73 m2 is achieved. In yet other exemplary embodiments, a reduction of at least 0.50 mL/min/1.73 m2 is achieved. In still other exemplary embodiments, a reduction of at least 0.75 mL/min/1.73 m2 is achieved.

In another aspect, a method of treating ADPKD according to the present disclosure achieves a decrease in total kidney volume greater than the decrease achieved by a treatment method according to the ADPKD management.

In another aspect, a method of treating ADPKD according to the present disclosure achieves a slower increase in total kidney volume greater than the increase resulting from a treatment method according to the ADPKD management.

In yet another aspect, a method of treating ADPKD according to the present disclosure results in a TKV over about 1 year of treatment, over about 3 years of treatment, or over about 5 years of treatment such that the TKV is at most 95% the value of TKV associated with treatment according to the ADPKD management over an equivalent period of time. In other embodiments, the TKV resulting from the inventive method is at most 94%, or at most 93%, or at most 92%, or at most 91%, or at most 90%, or at most 89%, or at most 88%, or at most 87%, or at most 86%, or at most 85% the value of TKV associated with treatment according to the ADPKD management over an equivalent period of time.

### Example

As discussed above, the FDA required postmarketing clinical trials following the approval of tolvaptan for the treatment of ADPKD to assess the potential for interaction at breast cancer resistance protein (BCRP), organic anion transport poly-peptide (OATP)1B1, and organic anion transporter (OAT)3 transporters.

The highest approved dose regimen is 90 mg in the morning followed by a 30 mg dose given 8-9 hours later. The half-life of tolvaptan is about 3.5 hours, so there is no accumulation and steady-state concentrations are achieved on day 1. Tolvaptan has pH independent solubility of about 0.01 g/250 ml. However, tolvaptan tablets contain tolvaptan formulated using a spray-dried technique, which may increase tolvaptan in vitro solubility up to sevenfold. The oxobutyric acid metabolite of tolvaptan (DM-4103) is formed by CYP3A4-mediated metabolism of another tolvaptan metabolite; consequently, plasma concentrations are less than 100 ng/ml for the first 2 hours following a 90 mg dose (data on file). The elimination half-life is ˜180 hours, so steady-state concentrations are achieved ˜8 weeks after the start of tolvaptan dosing. In vitro studies indicated that tolvaptan is a BCRP inhibitor, whereas the oxobutyric acid metabolite (DM-4013) is an inhibitor of (OATP)1B1 and (OAT)3.

In 2020, the FDA finalized a guidance document intended to help drug developers determine the in vivo drug-drug interaction (DDI) potential of an investigational drug product. The earlier draft version released in 2017 contained almost identical language with regard to the prediction of the potential for in vivo inhibition at BCRP, (OATP)1B1, and (OAT)3 transporters. The FDA guidance is also consistent with guidelines published by the European Medicines Agency in 2013 [Guideline on the Investigation of Drug Interactions, European Medicines Agency, 21 Jun. 2012, CPMP/EWP/560/95/Rev. 1 Corr. 2**, Committee for Human Medicinal Products (CHMP)—finalized 2013. <https://www.ema.europa.eu/en/documents/scientific-guide line/guide line-investigationdrug-interactions-revision-1_en.pdf.] and the Ministry of Labor and Welfare in Japan in 2018 [Ministry of Labor and Welfare. Guideline on drug interaction for drug development and appropriate provision of information, notification No. 0723-4, pharmaceutical evaluation division, pharmaceuticals safety and environmental Health bureau, Japan. Jul. 23, 2018. https://www.pmda.gajp/files/00022 8122.pdf. Jan. 20, 2021.].

Based on the FDA website “Drug Development and Drug Interactions; Table of Substrates, Inhibitors and Inducers,” rosuvastatin is considered to be a suitable substrate for use in clinical DDI studies for determining interactions at BCRP and (OATP)1B1. Other statins are also known to be substrates of (OATP)1B1. Furosemide is considered to be a suitable substrate for use in clinical DDI studies for determining interactions at (OAT)3.

In the phase III trials for ADPKD, statin use was unrestricted and no differences in statin use (duration, dose change, statin change, and permanent discontinuation) or incidences of statin-related adverse events were observed for subjects taking tolvaptan alone or taking tolvaptan plus statins (atorvastatin, fluvastatin, lovastatin, pitavastatin, pravastatin, rosuvastatin, simvastatin, and simvastatin/ezetimibe). In phase II and III trials for congestive heart failure, although tested daily tolvaptan doses may have been lower and treatment durations shorter than those used in ADPKD trials, DM-4103 concentrations at the minimum level predicted to be inhibitory at (OAT)3 were achieved (data on file). Furosemide was used by greater than 80% of congestive heart failure subjects enrolled in tolvaptan clinical trials (data on file) and the incidence of adverse events was similar for subjects taking tolvaptan+furosemide compared with placebo+furosemide. Despite the lack of an interaction based on clinical data, the FDA required postmarketing clinical trials.

The primary objective of Trial 1 was to determine the potential inhibitory effect of tolvaptan on the BCRP transporter substrate (i.e., rosuvastatin), and the potential inhibitory effect of the tolvaptan DM-4103 metabolite on an (OATP)1B1 transporter substrate (i.e., rosuvastatin). The primary objective of Trial 2 was to determine the potential inhibitory effect of DM-4103 on an (OAT)3 substrate (i.e., furosemide).

BCRP: Prazosin transport was evaluated in MDCKII BCRP-expressing cells (obtained from SOLVO Biotechnology) precultured for six to eight days. Mannitol transport was unaffected by tolvaptan at 10 μM. Ko143 hydrate, a BCRP inhibitor, decreased prazosin transport by 96.8%. tolvaptan concentrations of 0.10, 0.30, 1.0, 3.0, and 10 μM were tested.

(OAT)3: Estrone 3-sulfate transport was evaluated in HEK293 cells stably expressing human (OAT)3 (kit with ready to use 24 well plates were obtained from GenoMembrane). Probenecid, 30 μM, inhibited estrone 3-sulfate transport by 91.1%. DM-4103 concentrations of 0.064, 0.32, 1.6, 8.0, 40, and 200 μM were tested.

(OATP)1B1: Estradiol 17β-D-glucuronide transport was evaluated in HEK293 cells stably expressing human (OATP)1B1 (kit with ready to use 24 well plates were obtained from GenoMembrane). Rifampicin, 10 μM, inhibited estradiol 17β-D-glucuronide transport by 91.1%. DM-4103 concentrations of 0.064, 0.32, 1.6, 8.0, 40, and 200 μM were tested.

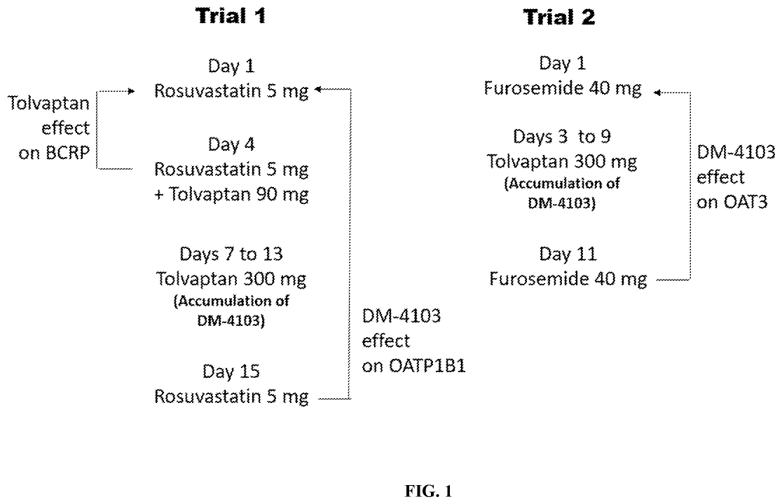

FIG. 1 illustrates the treatments administered in each of Trial 1 and Trial 2, as well as the comparisons that were made.

### Trial 1 Design

Trial 1 was designed as an open-label, 3-period, sequential crossover. Screening was conducted in the 28 days prior to dosing. Subjects checked into the clinic on day 1 and remained in-clinic for the duration of the study. On day 1, 5 mg rosuvastatin calcium was administered alone. On day 4, 5 mg rosuvastatin calcium was co-administered with 90 mg tolvaptan. On days 7 to 13, 300 mg tolvaptan was administered q.d. On day 15, 5 mg rosuvastatin calcium was administered alone. Subjects were discharged from the clinic on day 18 and a follow-up telephone call was conducted on day 22.

Doses on pharmacokinetic (PK) sampling days were administered in the fasted state with 240 ml of room temperature still water. No food was allowed from 10 h prior to dosing until lunch was given following the 4 h post-dose assessments; water was available ad libitum except for +/−2 h around dosing. On non-PK sampling days, tolvaptan was administered at least 30 min prior to breakfast. Safety was evaluated by laboratory values, vital signs, and 12-lead electrocardiogram (ECG) at screening, day 1, and discharge (no ECG), and adverse event reports.

The 5 mg dose of rosuvastatin calcium was chosen based on the high predicted value for a BCRP interaction and that increases in rosuvastatin maximal peak plasma concentration (Cₘₐₓ) up to 11-fold have been observed. A 90 mg dose of tolvaptan was chosen as it is the highest dose administered at any given time in the treatment of ADPKD. The FDA required that DM-4103 concentrations tested in this trial be similar to those observed at steady-state in ADPKD trials (˜7500 ng/ml), therefore, tolvaptan doses of 300 mg were administered for 7 days. Previously, 300 mg tolvaptan administered for 5 days to healthy subjects was shown to be safe and well-tolerated. After 48 hours, tolvaptan would be completely eliminated due to its short half-life, leaving only DM-4103 concentrations present in the plasma.

### Trial 2 Design

Trial 2 was designed as an open-label, 2-period, sequential crossover. Screening was conducted in the 28 days prior to dosing. Subjects checked into the clinic on day 1 and remained in-clinic for the duration of the study. On day 1, 40 mg furosemide was administered alone. On days 2 to 8, 300 mg tolvaptan was administered q.d. On day 10, 40 mg furosemide was administered alone. Subjects were discharged from the clinic on day 11 and a follow-up telephone call was conducted on day 17.

Trial 2 treatments were administered and safety assessed as described above for Trial 1. A 40 mg dose of furosemide was chosen as it was expected that furosemide concentrations would be measurable up to 24 hours and that this dose would have at least a twofold safety margin, as up to 80-mg doses have been administered safely to healthy subjects.

### Sample Collection Methods

For Trial 1, blood samples for rosuvastatin (4 ml using dipotassium ethylenediaminetetraacetic acid [K₂EDTA] as anti-coagulant) were taken at pre-dose and 0.5, 1, 2, 3, 4, 6, 8, 12, 24, 32, 48, 56, and 72 h post-dose on days 1, 4, and 15. Samples for tolvaptan (4 ml with sodium heparin as anticoagulant) were taken at 2 and 3 h post-dose on day 4. Samples for DM-4103 (4 ml with sodium heparin as anticoagulant) were taken at pre-dose and 24, 48, 72, 96, and 120 hours post-dose on day 13 (i.e., seventh day of dosing).

For Trial 2, blood samples for furosemide (4 ml using K₂EDTA as anticoagulant) were taken at pre-dose, 0.5, 1, 1.5, 2, 3, 4, 6, 8, 12, and 24 h on days 1 and 10. Samples for DM-4103 (4 ml with sodium heparin as anticoagulant) were taken at pre-dose, 24, 48, and 72 hours on day 8 (i.e., seventh day of dosing).

Plasma was evenly pipetted into 2 aliquots and stored at −70° C. or colder (rosuvastatin, tolvaptan, and DM-4103) or −20° C. or colder (furosemide) until analyzed.

### Analysis

Plasma rosuvastatin concentrations were determined using a specific and validated reversed-phase high performance liquid chromatography with tandem mass spectrometric detection (HPLC-MS/MS). Rosuvastatin and the internal standard rosuvastatin-d6 were extracted from human plasma containing K2EDTA as an anticoagulant using solid phase extraction. The method was validated for concentrations ranging from 0.500 to 300 ng/ml for rosuvastatin. All validation work and sample analysis were performed at ICON Laboratory Services, Inc. (Whitesboro, N.Y.).

The assay used to determine plasma tolvaptan concentrations also included DM-4103 as a standard. The precursor→production was m/z 479→252. DM-4103 concentrations were linear over the range of 12.5 to 12,500 ng/ml. All validation work and sample analysis were performed at ICON Laboratory Services, Inc. (Whitesboro, N.Y.).

Plasma furosemide concentrations were determined using a specific and validated reversed-phase HPLC-MS/MS method. Furosemide and the internal standard, furosemide-d5, were extracted from human plasma containing K2EDTA by protein precipitation. The method was validated for concentrations ranging from 5.00 to 5000 ng/ml. All validation work and sample analysis were performed at Altasciences (Quebec, Canada).

Rosuvastatin and furosemide concentrations were analyzed using noncompartmental methods (SAS version 9.4). For calculation of descriptive statistics and PK parameters, plasma concentration values below the lower limit of quantitation (LLOQ) prior to first measurable concentrations were imputed to 0. Concentration values below the LLOQ following the last sample with a measurable concentration were excluded from the analyses. Actual blood sample times post-dose were determined and reported as 2 decimals. Values for Cₘₐₓ and the time of maximal plasma concentration (Tₘₐₓ) were determined directly from the observed data. Values of area under the concentration-time curve from time 0 to the time of the last measurable concentration (AUCₜ) were estimated using the linear trapezoidal rule.

The terminal-phase elimination rate constant (λ_{z}) was estimated by a log-linear regression of at least three non-zero concentrations; regressions with r² values less than 0.8 were not reported. The terminal-phase elimination half-life (t_{1/2, z}) was determined as (ln 2)/λ_{z}. Values of AUC from time 0 to infinity (AUCₜ) were determined as AUCₜ+Cₗₐₛₜ/λ_{z}. The value of apparent clearance from plasma following extravascular drug administration (CL/F) was determined as dose/AUC_{∞}.

Descriptive statistics were presented by treatment and analyte. A maximum of three significant figures were used for table presentations except for Tₘₐₓ, which was reported to two decimals and t_{1/2,z}, which was reported to one decimal.

The geometric mean ratios with 90% confidence intervals (CIs) of Cₘₐₓ, AUCₜ, and AUC_{∞} were determined for rosuvastatin with tolvaptan and rosuvastatin in the presence of DM-4103 compared with rosuvastatin alone (periods 2 or 3 compared with period 1) and for furosemide in the presence of DM-4103 compared with furosemide alone (period 3 compared with period 1). An analysis of variance with a factor of period and random effect of subjects was performed on the natural-log transformed PK parameters using the MIXED procedure in SAS. From each analysis, the least square means, their difference, and the 90% CI for their difference were derived. Then, the anti-logs of the difference and the confidence limits provided the estimate and 90% CI for the ratio of the geometric means of the test treatment to the reference treatment. Only subjects with values in both treatments were used in the analysis.

### Results

Tolvaptan inhibition of prazosin transport in BCRP overexpressing MDCKII cells provided a half-maximal inhibitory concentration (IC₅₀) estimate of 8.32 μM. The FDA guidance predicts an interaction when “ . . . drug is administered orally, and the I_{gut}/IC₅₀ or Kᵢ≥10 where I_{gut}=dose of inhibitor/250 ml.” A 90 mg dose of tolvaptan is 200 μmol, which results in a predicted Ki=800/8.32=96.4, indicating potential for an interaction. DM-4103 inhibition of estradiol 17β-D-g lucuronide transport in (OATP)1B1 overexpressing HEK293 cells provided an IC₅₀ estimate of 0.255 μM. The FDA guidance states that potential for a clinically relevant interaction exists if R=1+([f_{u,p}×|_{in,max}]/IC₅₀) greater than or equal to 1.1 where R is the predicted ratio of the victim drug's AUC in the presence and absence of the investigational drug as the inhibitor, f_{u,p} is the unbound fraction in plasma, IC₅₀ is the half-maximal inhibitory concentration, and I_{in,max} is the estimated maximum plasma inhibitor concentration at the inlet to the liver. Considering uncertainties in the protein binding measurements, the unbound fraction (f_{u,p}) should be set to 1% if experimentally determined to be less than 1%. As a metabolite formed slowly by metabolism after absorption of tolvaptan, DM-4013 has no intestinal availability and portal vein concentrations are negligible compared with circulating concentrations at steady-state, so I_{in,max} was set equal to the circulating concentrations of DM-4103.

Following the 90+30 mg dose regimen of tolvaptan in subjects with ADPKD, mean DM-4103 concentrations were ˜7500 ng/ml or 15.7 μM (data on file). DM-4103 is greater than 99.8% plasma protein bound so f_{u,p} was set to 0.01. Consequently, for DM-4103, R=1+([0.01×15.7]/0.255)=1.62, indicating potential for an interaction. DM-4103 inhibition of estrone 3-sulfate transport in (OAT)3 overexpressing HEK293 cells provided an IC₅₀ estimate of 0.425 μM. The FDA guidance predicts an interaction if “ . . . the I_{max,n}/IC₅₀ value is ≥0.1.” For DM-4103, 15.7*0.01/0.425=0.37, indicating potential for an interaction. The ratio is equal to 0.1 when DM-4103 concentration is ˜2034 ng/ml. For Trial 1, 16 subjects were enrolled and 14 completed. One subject was discontinued due to a treatment-emergent adverse event (TEAE) of palpitations at 70 hours following rosuvastatin dosing on day 1. One subject withdrew following rosuvastatin dosing on day 1. For Trial 2, 14 subjects were enrolled and completed. The demographic characteristics of enrolled subjects are summarized in Table 1.

TABLE 1

| | Trial 1 | | Trial 2 | |
|---|---|---|---|---|
| Age, mean (range) | 27.3 | (20-42) | 28.1 | (19-43) |
| Weight, mean (range) | 70.8 | (48.5-92.4) | 70.3 | (53.7-87.2) |
| Sex, n (%) | | | | |
| Male | 10 | (62.5) | 7 | (50.0) |
| Female | 6 | (37.5) | 7 | (50.0) |
| Race, n (%) | | | | |
| White | 13 | (18.3) | 12 | (85.7) |
| Black | 2 | (12.5) | 1 | (7.1) |
| American Indian or | 1 | (6.3) | 1 | (7.1) |
| Alaskan Native | | | | |
| Ethnicity, n (%) | | | | |
| Hispanic or Latino | 1 | (6.3) | 4 | (28.6) |
| Not Hispanic or Latino | 15 | (93.8) | 10 | (71.4) |

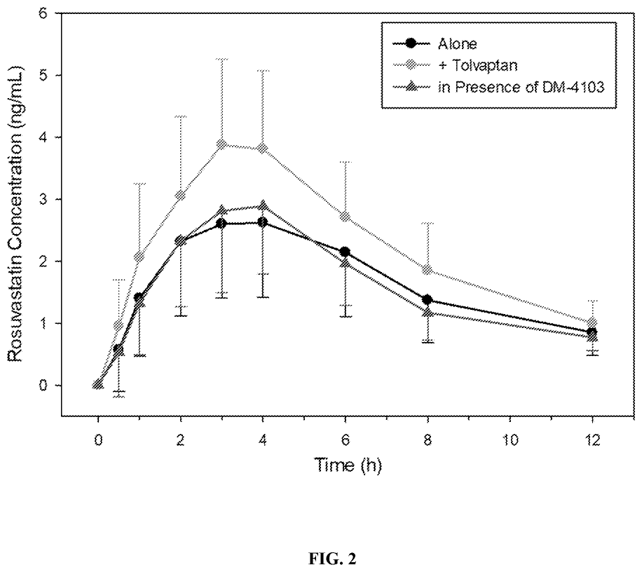

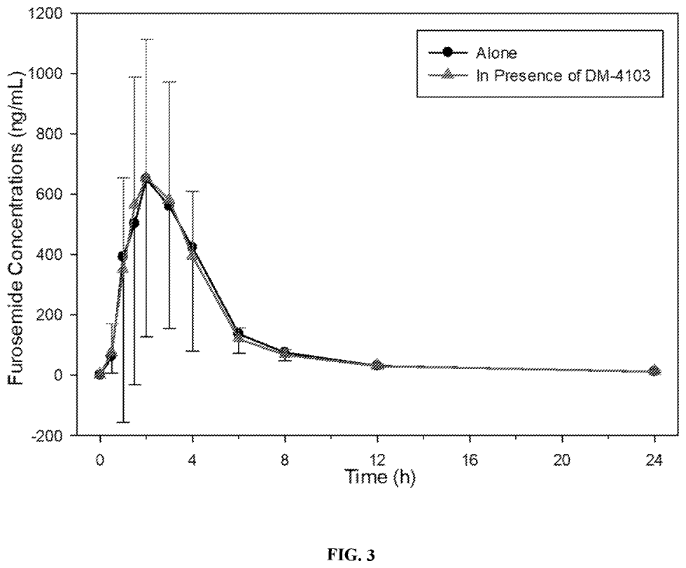

Plots of mean (SD) rosuvastatin and furosemide plasma concentrations are presented in FIGS. 2 and 3, respectively. PK parameters for rosuvastatin and furosemide are presented in Tables 2 and 3, respectively.

FIG. 2 shows the mean (SD) rosuvastatin plasma concentrations after administration of 5 mg rosuvastatin calcium alone, with 90 mg of tolvaptan or 48 h following 7 days of 300 mg tolvaptan tolvaptan q.d. (i.e., in the presence of DM-4103) to 14 healthy adult subjects. No clinically-relevant inhibition of BCRP by tolvaptantolvaptan and no inhibition of OAPT1B1 by DM-4103 was observed.

FIG. 3 shows the mean (SD) furosemide plasma concentrations after administration of 40 mg furosemide alone or 48 hours following 7 days of 300 mg tolvaptan q.d. (i.e., in the presence of DM-4103) to 14 healthy subjects. No inhibition of OAT3 by DM-4103 was observed.

TABLE 2
Mean (SD) plasma rosuvastatin PK parameters in 14 subjects

| | | | 5 mg | | 5 mg | |
|---|---|---|---|---|---|---|
| | 5 mg | | rosuvastatin + | | ROS in | |
| Param- | rosuvastatin | | 90 mg | | presence of | |
| eter | alone | | tolvaptan | | DM-4103 | |
| Cₘₐₓ, | 2.81 | (1.27) | 4.01 | (1.30) | 3.03 | (1.20) |
| ng/ml | | | | | | |
| Tₘₐₓ, h | 3.50 | (2.00-6.00)* | 3.03 | (2.00-4.12)* | 4.00 | (3.00-6.00)* |
| AUCₜ, | 18.9 | (10.1) | 29.7 | (14.6) | 18.4 | (8.26) |
| ng*h/ml | | | | | | |

| | | | | | |
|---|---|---|---|---|---|
| t_{1/2, z}, h | 2.4-4.3** | 4.5 | (1.5)*** | 3.6 | (0.6)**** |
| AUC_{∞}, | 20.3-36.0** | 38.0 | (14.6)*** | 27.2 | (8.21)**** |
| ng*h/ml | | | | | |
| CL/F, | 28.5-68.2** | 36.6 | (16.4)*** | 44.3 | (16.6)**** |
| ml/ | | | | | |
| min/kg | | | | | |

*median (minimum-maximum)
**values are minimum-maximum, n = 7
***n = 11
****n = 8

As can be seen in Table 2, administration of rosuvastatin alone

TABLE 3
Mean (SD) plasma furosemide PK parameters in 14 subjects

| | | |
|---|---|---|
| | 40 mg furosemide | 40 mg furosemide in |
| Parameter | alone | presence of DM-4103 |

| | | | | |
|---|---|---|---|---|
| Cₘₐₓ, ng/ml | 992 | (580) | 873 | (475) |
| Tₘₐₓ, h | 2.00 | (1.00-400)* | 2.00 | (1.00-400)* |
| AUCₜ, ng*h/ml | 2920 | (1400) | 2900 | (1140) |
| AUC_{∞}, ng*h/ml | 2950 | (1390)** | 3070 | (1150)** |
| t_{1/2, z}, h | 6.1 | (2.4)** | 6.7 | (1.3)** |
| CL/F, ml/min/kg | 3.88 | (1.55)** | 3.53 | (1.12)** |

*median (minimum-maximum)
**n = 13

Geometric mean ratios and 90% CIs are presented in Table 4.

TABLE 4

| Comparison | Cₘₐₓ | AUCₜ | AUC_{∞} |
|---|---|---|---|
| BCRP inhibition | | | |
| Rosuvastatin + tolvaptan | 1.538 | 1.691 | 1.281* |
| vs. rosuvastatin alone, | 1.255-1.883 | 1.335-2.141 | 1.148-1.429 |
| n = 14 | | | |
| (OATP)1B1 inhibition | | | |
| Rosuvastatin in presence | 1.129 | 1.045 | 0.998** |
| of DM-4103 vs. rosuvastatin | 0.995-1.335 | 0.881-1.239 | 0.876-1.138 |
| alone, n = 14 | | | |
| (OAT)3 inhibition | | | |
| Furosemide in presence | 0.907 | 1.04 | 1.018*** |
| of DM-4103 vs. furosemide | 0.794-1.035 | 0.938-1.152 | 0.916-1.131 |
| alone, n = 14 | | | |

*number of subjects with parameter in both treatment periods, n = 6
**number of subjects with parameter in both treatment periods, n = 5
***number of subjects with parameter in both treatment periods, n = 12

Tolvaptan administration concomitant with rosuvastatin administration was confirmed as mean (SD) concentrations at 2 and 3 hours post-dose were 422 (241) and 409 (184) ng/ml, respectively.

Mean (SD) DM-4103 concentrations at 48 hours post-dose (i.e., pre-dose of the last dosing period) were 7240 (1880) ng/ml and 6430 (2030) ng/ml in trial 1 and trial 2, respectively, well above the predicted inhibitory levels.

For Trial 1, treatment-emergent adverse events (TEAEs) reported in at least two subjects/period were, in period 1 (rosuvastatin), nausea (12.5%), in period 2 (rosuvastatin+tolvaptan), pollakiuria (35.7%), thirst (50%), dysgeusia (14.3%), and headache (14.3%), in period 3 (tolvaptan alone), pollakiuria (71.4%), thirst (41.9%), nausea (21.4), and constipation and dry mouth (both 14.3%), and in period 3 (rosuvastatin in presence of DM-4103), headache (14.3%).

For Trial 2, TEAEs reported in at least two subjects/period were, in period 1 (furosemide alone), polyuria (64.3%), in period 2 (tolvaptan alone), polyuria (100%), thirst (92.9), dry mouth (21.4%), constipation, dizziness, headache, and insomnia (all 14.3%) and, in period 3 (furosemide in presence of DM-4103), and headache (21.4%).

All incidences of pollakiuria, polyuria, dry mouth, and thirst were considered to be related to tolvaptan. No subjects had adverse events related to changes in laboratory values or vital signs.

Rosuvastatin Cₘₐₓ and AUCₜ were increased 1.54- and 1.69-fold, respectively, when administered with 90 mg tolvaptan. Table 4 in the prescribing information for CRESTOR indicates that interactions are only considered clinically meaningful if the increases in AUC are at least 1.9-fold and the increases in Cₘₐₓ are at least 2.2-fold. Therefore, the increases observed in this trial would not be considered clinically meaningful.

Based on the guidances for prediction of potential for interaction at BCRP, it is assumed the dose is completely soluble in 250 ml; a 90-mg tolvaptan dose (200 μmol) in 250 ml has a predicted concentration of 800 μM. However, tolvaptan has pH independent solubility of about 0.01 mg/250 ml (˜0.09 μM). Even if tolvaptan solubility was increased sevenfold, due to spray-drying, tolvaptan would not be predicted to have clinically relevant interactions at BCRP transporters.

A potential mechanism behind the observed interaction could be tolvaptan inhibition of the sodium-dependent tauro-cholate co-transporting polypeptide (NTCP). It has been reported that rosuvastatin is transported by NTCP with human hepatocyte studies suggesting that NTCP alone could account for ˜35% of rosuvastatin uptake. Tolvaptan was shown to be an inhibitor of NTCP, although the IC₅₀ value was higher than Cₘₐₓ. However, liver concentrations of tolvaptan were predicted to be much higher than blood concentrations (liver:blood ratio 38.76) and tolvaptan was shown to accumulate at least 10-fold in sandwich-cultured human hepatocytes; “The total cellular concentration of tolvaptan ranged from ˜2 μM to 500 μM and was greater than the respective incubation concentration (0.15-50 μM).” Thus, liver concentrations of tolvaptan may be reaching inhibitory levels.

As shown in Table 4, the lower bounds of the 90% CI for Cₘₐₓ and AUCₜ are less than 1 for both rosuvastatin in the presence of DM-4103 compared with rosuvastatin alone and furosemide in the presence of DM-4103 versus furosemide alone, therefore we can conclude that DM-4103 is not an inhibitor of (OATP)1B1 or (OAT)3 at clinically-relevant concentrations. For (OATP)1B1 and (OAT)3, the FDA guidance instructs that fraction unbound for plasma protein binding less than 1% be set to 1% “considering uncertainties in protein binding measurements.”

DM-4103 protein binding was determined to be greater than 99.8% using a micropartition device (Centrifree YM-30) followed by HPLC-MS/MS detection of concentrations in the ultrafiltrate. The effect of six other highly plasma protein bound compounds on DM-4103 binding was also evaluated. No interactions were detected with DM-4103 binding greater than 99.8% in all samples (data on file). No potential for interaction at either transporter would have been predicted if a free fraction of 0.1% had been used, consistent with the clinical findings. These results support the use of drug substance solubility and actual unbound fraction in plasma in the predictions for clinically meaningful drug interactions.

1. A method for treating polycystic kidney disease in a patient taking breast cancer resistance protein (BCRP) substrate, comprising administering tolvaptan or a prodrug thereof to a polycystic kidney disease patient, wherein the BCRP substrate is selected from the group consisting of mitoxantrone, bisantrene, aza-anthrapyrazole (BBR3390), topotecan, SN-38, irinotecan, diflomotecan, methotrexate, methotrexate-glu2, methotrexate-glu3, AZT, AZT 5′-monophosphate, lamivudine (3TC), prazosin, indolocarbazole, flavopiridol, canertinib (CI1033), imatinib mesylate (STI571), gefitinib (ZD1839), nilotinib, glyburide, cimetidine, sulfasalazine, nitrofurantoin, rosuvastatin, pantoprazole, carcinogen 2-amino-1-methyl-6-phenylimidazo[4,5-b]pyridine (PhIP), protoporphyrin ix, the lipid phosphatidylserine, and flavonoids.

2. (canceled)

3. The method according to claim 1, wherein the BCRP substrate is rosuvastatin.

4. The method according to claim 3, wherein, when the patient is administered 5 mg rosuvastatin, the maximum rosuvastatin concentration (Cₘₐₓ) increases by about 1.50- to 1.60-fold and the area under the curve (AUC) for rosuvastatin increases by about 1.65- to 1.75-fold, when the patient is administered 90 mg tolvaptan or 48 hours following seven (7) days of once-daily 300 mg tolvaptan.

5. The method according to claim 1, wherein the effect of up to 90 mg tolvaptan on the BCRP substrate is not clinically meaningful.

6. The method according to claim 1, wherein the effect of up to once-daily 300 mg tolvaptan on the BCRP substrate is not clinically meaningful.

7. A method for treating polycystic kidney disease in a patient taking organic anion transport polypeptide substrate, comprising administering tolvaptan or a prodrug thereof to a polycystic kidney disease patient.

8. The method according to claim 7, wherein the organic anion transport polypeptide substrate is an (OATP)1B1 substrate selected from the group consisting of ambrisentan, asunaprevir, atorvastatin, atrasentan, axitinib, belantamab mafodotin, belzutifan, benzylpenicillin, bosentan, brincidofovir, caspofungin, cerivastatin, cholecystokinin, cholic acid, clotrimazole, cobimetinib, conjugated estrogens, cyclosporine, digoxin, dinoprostone, elagolix, eluxadoline, empagliflozin, enalapril, erythromycin, ezetimibe, fexofenadine, fimasartan, fluvastatin, gadoxetic acid, gimatecan, glecaprevir, grazoprevir, letermovir, levomenol, levosalbutamol, liothyronine, liotrix, lovastatin, methotrexate, mycophenolate mofetil, olmesartan, ouabain, paritaprevir, penicillamine, pitavastatin, prasterone, pravastatin, raloxifene, remdesivir, repaglinide, revefenacin, rosuvastatin, selexipag, simeprevir, simvastatin, sumatriptan, taurocholic acid, technetium tc-99 m mebrofenin, temocapril, tenofovir alafenamide, torasemide, ubrogepant, valsartan, and voxilaprevir.

9. (canceled)

10. The method according to claim 7, wherein the organic anion transport polypeptide substrate is an (OATP)1B3 substrate selected from the group consisting of ambrisentan, atogepant, atorvastatin, belantamab mafodotin, belzutifan, bempedoic acid, brincidofovir, caspofungin, cholecystokinin, cholic acid, cobimetinib, conjugated estrogens, docetaxel, empagliflozin, erythromycin, fexofenadine, fluvastatin, gadoxetic acid, glecaprevir, grazoprevir, letermovir, levomenol, levosalbutamol, liothyronine, liotrix, methotrexate, mycophenolate mofetil, olmesartan, opicapone, ouabain, paclitaxel, parachlorophenol, paritaprevir, pitavastatin, prasterone, pravastatin, raloxifene, revefenacin, rifampicin, romidepsin, selexipag, simeprevir, sincalide, taurocholic acid, technetium tc-99 m mebrofenin, tenofovir alafenamide, testosterone, trastuzumab deruxtecan, ubrogepant, valsartan, and voxilaprevir.

11. (canceled)

12. The method according to claim 7, wherein the organic anion transport polypeptide substrate is rosuvastatin.

13. The method according to claim 12, wherein, when the patient is administered 5 mg rosuvastatin in the presence of tolvaptan metabolite (DM-4103), the lower bounds of the 90% confidence interval for maximum concentration (Cₘₐₓ) and area under the curve (AUC) is less than about 1.0% for rosuvastatin in the presence of tolvaptan metabolite (DM-4103) as compared to the lower bounds of the 90% confidence interval for maximum concentration (Cₘₐₓ) and area under the curve (AUC) when rosuvastatin is administered alone.

14. The method according to claim 7, wherein the effect of up to 90 mg tolvaptan on the organic anion transport polypeptide substrate administered is not clinically meaningful.

15. The method according to claim 7, wherein the effect of up to once-daily 300 mg tolvaptan on the organic anion transport polypeptide substrate administered is not clinically meaningful.

16. A method for treating polycystic kidney disease in a patient taking an organic anion transporter (OAT)3 substrate, comprising administering tolvaptan or a prodrug thereof to a polycystic kidney disease patient, wherein the (OAT)3 substrate is selected from the group consisting of acamprosate, acyclovir, allopurinol, alprostadil, aminohippuric acid, avibactam, baricitinib, benzylpenicillin, betamethasone phosphate, budesonide, bumetanide, captopril, cefacetrile, cefaclor, cefaloridine, cefazolin, cefdinir, cefotiam, ceftibuten, ceftizoxime, cephalexin, cholic acid, cimetidine, citrulline, clofarabine, cloxacillin, conjugated estrogens, cortisone acetate, cyclic adenosine monophosphate, dexamethasone, dexamethasone acetate, dinoprostone, doripenem, edaravone, ellagic acid, eluxadoline, empagliflozin, estradiol, estrone, famotidine, fexofenadine, fluorescein, glutaric acid, hydrochlorothiazide, hydrocortisone, hydroflumethiazide, indomethacin, leucovorin, levocarnitine, medrysone, mercaptopurine, methotrexate, oseltamivir, oxalic acid, oxytetracycline, pemetrexed, piperacillin, polythiazide, pravastatin, prednisolone phosphate, quinapril, ranitidine, relebactam, rosuvastatin, saxagliptin, silibinin, sitagliptin, succinic acid, taurocholic acid, tazobactam, tenofovir, tenofovir alafenamide, tenofovir disoproxil, tetracycline, trifluridine, valaciclovir, valproic acid, and zidovudine.

17. (canceled)

18. The method according to claim 16, wherein the (OAT)3 substrate is furosemide.

19. The method according to claim 18, wherein, when the patient is administered 40 mg furosemide in the presence of tolvaptan metabolite (DM-4103), the lower bounds of the 90% confidence interval for maximum concentration (Cₘₐₓ) and area under the curve (AUC) is less than one (1) percent for furosemide in the presence of DM-4103 (one-daily 300 mg tolvaptan) as compared to the lower bounds of the 90% confidence interval for maximum concentration (Cₘₐₓ) and area under the curve (AUC) when furosemide is administered alone.

20. The method according to claim 16, wherein the effect of up to once-daily 300 mg tolvaptan on the (OAT)3 substrate is not clinically meaningful.

21. A method comprising administering to a patient in need thereof a clinically meaningful amount of tolvaptan and a clinically meaningful amount of a substrate selected from BCRP substrates, (OAT)3 substrates, (OATP)1B1 substrates, and (OATP)1B3 substrates.

22. The method according to claim 21, wherein the substrate is an (OAT)3 substrate, an (OATP)1B1 substrate, or an (OATP)1B3 substrate, and wherein the tolvaptan and the substrate are co-administered.

23. The method according to claim 21, wherein the substrate is a BCRP substrate, and the tolvaptan and the BCRP substrate are administered no more than ten hours apart.
